(19) United States
(12) Patent Application Publication
(10) Pub. No.: US 2014/0114146 A1
(43) Pub. Date: Apr. 24, 2014

(54) DIAGNOSTIC DEVICES, MANDIBULAR MANIPULATORS WITH TONGUE AND NASAL SENSORS, AUTOMATED MANDIBULAR MANIPULATORS AND RELATED METHODS

(71) Applicant: Kosmo Technologies, LLC, Salt Lake City, UT (US)

(72) Inventors: William H. Hanewinkel, Salt Lake City, UT (US); Michael B. Gleeson, Salt Lake City, UT (US); Jeremy D. Andra, Draper, UT (US); Ellen Crean, Phoenix, AZ (US)

(73) Assignee: Kosmo Technologies, LLC, Salt Lake City, UT (US)

(21) Appl. No.: 14/060,535

(22) Filed: Oct. 22, 2013

Related U.S. Application Data
(60) Provisional application No. 61/716,750, filed on Oct. 22, 2012.

(51) Int. Cl. A61B 5/00; A61B 5/08; A61B 5/087; A61B 5/04; A61B 5/097

(52) U.S. Cl. CPC A61B 5/4552; A61B 5/04001; A61B 5/097; A61B 5/087; A61B 5/0826
USPC 600/301; 600/590; 600/547

(57) ABSTRACT

Diagnostic devices may include features for determining at least one of bilateral nasal flow and tongue position. Automated mandibular manipulators may include at least one motor including a feedback feature for communicating at least one position of at least a portion of the mandibular manipulator to a computer.

### CROSS-REFERENCE TO RELATED APPLICATIONS

This application claims the benefit of U.S. Provisional Patent Application Ser. No. 61/716,750, filed Oct. 22, 2012, entitled “Diagnostic Devices, Mandibular Manipulators with Tongue and Nasal Sensors, Automated Mandibular Manipulators and Related Methods,” the disclosure of which is incorporated herein by reference in its entirety.

### TECHNICAL FIELD

This disclosure is generally related to the field of medical devices and methods such as, for example, medical devices and methods for sleep apnea diagnoses and devices for use with sleep apnea diagnoses.

### BACKGROUND

Recent studies performed and published by doctors at Walter Reed Hospital indicate that Oral Appliance Therapy (OAT) can be used as a first line medical treatment of sleep apnea in patients that present with mild to moderate cases. In comparison to more invasive (surgical) or inconvenient/uncomfortable therapies (CPAP), OAT is comfortable, performs well, and allows the patient the convenience of travel or to be in places without reliable electrical power.

Other recent published studies indicate that the placement of the tongue during sleep and nasal flow are new metrics that should be recorded in sleep studies along with the other physiological measurements currently being recorded during the patient's study. The current physiological measurements include respiratory rate, blood pressure, blood oxygen content, electroencephalography (EEG), and body position.

The nasal airflow is a rather interesting indicator of brain activity during sleep. The airflow in each nasal passage can vary depending on the amount of brain activity either right or left. More brain activity on one side causes physical swelling of the nasal passage on that side. Consequently, this can cause a lower flow rate of air to the lungs with the results being lower blood oxygen content. This metric becomes critical to measure so that the attending physician can recognize normal versus abnormal bilateral nasal flow rate in comparison with something abnormal causing a blockage within the nasal cavity.

A contributing cause of obstructive sleep apnea can be the falling back of the tongue within the patient's airway. The addition of a device to sense tongue position during the patient's polysomnography testing would provide a needed metric. This metric would improve the physician's diagnostic data when evaluating a patient and determining treatment options.

### SUMMARY

Described is a device that can automatically manipulate the mandible in three dimensions while also sensing that the tongue is in its sleep position (e.g., up against the palate (e.g., the hard palate) behind the upper incisors). The device may include sensors that measure bilateral flow or pressure drop of air at the nasal openings. Such a mandibular manipulator is disclosed in U.S. Pat. No. 8,226,407, assigned to Kosmo Technologies, LLC, the disclosure of which is hereby incorporated herein in its entirety by this reference. Besides including the tongue and bilateral flow nasal sensors, this disclosure also describes the means useful within the patient's oral cavity for attaching the manipulator to the patient's teeth while providing comfortable space for the tongue. Generally, the oral cavity space needs to be as near normal as possible for tongue movement when the patient is in a sleep state. At least one aspect of novelty of this disclosure is that it enables the performance of a sleep study while manipulating the patient's mandible in at least two dimensions while he or she is asleep. In present sleep study testing, the patient needs to be awakened to allow adjustments to the mandible. In some embodiments, both the tongue placement and the nasal flow sensors could be electrical sensors however the means of performing these measurements could also be pneumatic type devices with the pneumatic measurements converted to electrical signals for reading by a digital or analog computer outside of the patient's body.

The fewer components within the oral cavity results in less brain arousal so that a more accurate sleep test can be conducted without disturbing the patient. In additional embodiments, sensing tongue placement in a noninvasive manner may comprise: 1) measuring the electrical capacitance of the oral tissues by placing conductive sensors on each external cheek surface with the sensors connected to a sensitive capacitance metering device, and 2) measuring the electrical potential of the tongue's extrinsic muscles (e.g., genioglossus muscle, protrudes the tongue as well as depressing its center; hyoglossus muscle, depresses the tongue; styloglossus muscle, elevates and retracts the tongue) with the sensors attached to the upper neck position on the patient. In the electrical capacitance measurement, the sensors could be also placed on the external nose surface with its corresponding sensor placed external to the lower chin. In some embodiments, the sensors for these applications may be similar to sensors sold by Cadwell Laboratories of Kennewick, Wash. (e.g., Cadwell Laboratories' sensor part number 302285-000).

Also disclosed is software operating on a digital computer. The computer is provided with feedback that represents all of the patient's recorded physiological data including tongue placement, nasal bilateral flow, and servo motor position for the mandible movement. After running this input data through the computer's software routine, its output drives the manipulator's motors in very small increments to adjust the patient's mandible vertically, in the anterior/posterior, and sagittally. The algorithm of the software is searching for the best position of the mandible using comparative input, feedback, and output to optimize as much of the physiological data as possible. The disclosure also enables the manipulation of the mandible manually through the interface of the digital computer.

In some embodiments, a portion of the manipulator (e.g., one or more motors) may provide feedback information regarding the amount of mandible muscle resistance from the subject's mandible. A computer may receive and analyze this feedback data from the motors to quantify and report the mandible muscle resistance from the subject's mandible.

While it is understood that there are several causes of sleep apnea, this device provides improved means of providing the doctor with information to determine which treatment options should be used. The treatment options can vary from OAT for mild to moderate cases of apnea to directing the use of CPAP or even invasive surgical means to correct the apnea.

Other mandibular manipulators, such as the MATRx™ device described in U.S. Patent Application Publication No. 2010/0316973, the disclosure of which is hereby incorporated herein in its entirety by this reference, only adjust the anterior/posterior position of the mandible. Studies have proven that sagittal, vertical, and anterior/posterior positions of the mandible relative to the maxilla are critical in producing the best oral appliance therapy that will do the long term least harm to the patient (temporomandibular joint disorder (TMD) injury). The addition of the physiological metrics of the tongue position sensor and nasal bilateral flow measurement will help provide the most complete picture to the medical practitioner during a sleep study.

In a ten subject pilot study performed by Kosmo Technologies discussed below, the results of opening up the oropharyngeal airway successfully in all ten apnea patients utilizing a manual titration of the mandible and measuring the oropharyngeal before and after the mandible adjustment are shown. These results illustrate the novelty of minutely manipulating the mandible in both the vertical and the anterior/posterior (A/P) position to improve the airway for sleep. The results show that each patient is unique in positioning their mandibles and that mandible positioning is advantageously performed in a manner that is both simultaneous in two directions (vertical and A/P) and in millimeter increments rather than in large centimeter increments. A contrary school of thought on this procedure of manipulating the mandible to open the airway utilizes only the A/P adjustment to the mandible. However, there are many contradictions demonstrating that this single mode of adjustment does not work on every patient. The pilot study indicates that every patient is unique and that it requires a custom blending of vertical and A/P positioning in minute movements to optimize the airway opening. Data also suggests that the oropharyngeal can be stretched both laterally and anterior/posterior when opening the airway using the two-directional method.

The Automated Mandibular Manipulator (AMM) Device is designed to move the mandible remotely and minutely in three axes: anterior-posterior, vertically, and sagittally while a patient is sleeping during a recorded sleep study. In one embodiment, the sagittal adjustment is made manually while the other two axes are adjusted by feedback and output of a digital computer. Typically, the sagittal position is usually preset to the patient's centerline teeth position and can be then left alone for the remainder of the study. All of the current physiological metrics being recorded in a sleep study will be utilized as feedback, and provide input to a computer and its software to precisely locate the mandible into the best position for the patient's treatment. These metrics would include respiratory rate, heart rate, EEG, body temperature, percentage of blood oxygen content, and EKG.

This device will also have the ability to record bilateral nasal flow and tongue position that are also important metrics to recognize for successful outcomes in treating obstructive sleep apnea. The AMM will be developed to work in both lab diagnostic sleep equipment and ambulatory sleep testing devices.

In constructing an oral appliance for treating sleep apnea for mild to moderate cases, it is first critical to fully understand the cause and effect of the apnea. Utilizing the AMM while monitoring the patient's physiological metrics will determine the exact mandible position in three axes that is successful for preventing sleep breathing disorder events. The mandible position can be recorded for the purpose of constructing an oral appliance. This new protocol will enable a sleep doctor to confidently find the optimal mandible position for an apnea patient while ensuring patient comfort prior to creating an oral appliance.

A currently marketed mandibular manipulator can be seen, for example, in U.S. Pat. No. 8,226,407. The mandibular manipulator can titrate the mandible manually using the rotating gear and rack mechanism in its design. This technique lends itself well to adapting miniature motors for driving the Anterior/Posterior (A/P) and Vertical motions. With a patient asleep and relaxed, very little motor torque will be required for adjusting the 2 positions less the sagittal. The motors will have integral gear heads to slow the output shaft rotation and amplify the mechanical torque and will either be small DC servos, stepper or piezoelectric motor types. The motors can be as small as 6 mm diameter and weigh a mere 1.4 grams. Another feature making the instant disclosure successful is having as low a weight as possible or in other terms causing as little disturbance to the patient so that they can sleep comfortably as possible without brain arousal during the sleep study.

Starting within the patient's mouth, there are two minimally sized plastic teeth trays that can be moved independent of one another. A mechanical feature on the anterior surface of the trays is used to lock the trays onto the drive mechanism located outside of the patient's mouth. A soft preformed acrylic material attached to the bite arches, both upper and lower, can be shaped to the patient's teeth both by the patient biting into the material but also by manually shaping the material by one of the medical team. Once this shape has been satisfactorily created, the bite arches can then be cured outside of the patient's mouth using a visible light of a specific electromagnetic wave frequency, such as a blue LED. Once the curing process has been performed, the device can be reinserted into the patient's mouth where in the bite arches now fit the teeth precisely in such a way that there is no relative motion between the teeth and bite arches.

In another embodiment, the acrylic material can be replaced by a “boil and bite” thermoplastic material. Both these methods create a compliant precise means of temporarily attaching the bite arches to the teeth without play or slippage. Integral to the upper teeth tray will be a means of remotely sensing whether the tongue is in a physiological rest position at the roof of the mouth while pressed forward or is falling to the floor of the mouth or is falling back into the throat. As another embodiment, the tongue placement sensor and bilateral nasal flow sensor can be utilized without the AMM.

Outside of the patient's mouth is the drive mechanism for creating forces used to titrate the mandible in very slow and small increments. Keeping the entire drive mechanism as small and lightweight as possible reduces arousal moments in the patient's sleep cycle. Mechanical features on the A/P and Vertical slides will be used to attach to the teeth trays. Integral to (e.g., attached to) the drive mechanism will be two bilateral sensors to either measure pressure or flow at the nasal passages. This nasal flow device can be integral to the exterior mechanical drive mechanism or something similar to a cannula that has a septum between nasal tubes. Either method uses a transducer to change the airflow data to electrical signals for the feedback to the digital computer.

The tongue position measurement may be integral to (e.g., attached to) the upper bite tray with a single or multiple tubular passageways within it terminating at the inner surface with an aperture that the tongue rests against. These passageways are brought out of the patient's mouth with very small lightweight tubing. The tubing holds either a small air pressure or small air vacuum. Electrical transducers change the pressure signal to an electrical signal for feedback to the digital computer.

Other physiological metrics are in some form of electrical signal and sent as feedback to the digital computer.

In some embodiments, the mechanism AMM for the described embodiment has the following adjustment characteristics. The travel in the Anterior-Posterior (A/P) direction is about 7 mm anterior and 5 mm posterior. Sagittal direction movement is about 3 mm left and right. The vertical travel is about 9 mm starting at about 2 mm incisor-to-incisor occlusion. In some embodiments, these values can be the minimal amount of motion that the manipulator is capable of traversing.

Having the capability to adjust the mandible in all three axes, along with sensing tongue position, and bilateral flow measurement combined with physiological metrics of the sleep study can provide the best feedback loop back to the AMM's motor drives through the digital computer's algorithm. Manipulating the patient's mandible in this manner, with slow incremental adjustments, can optimize the patient's mandible for sleep, and supply other critical information regarding the patient's apnea causal effects. Should the sleep study outcome show that the best treatment for the patient is an oral appliance, the physician has the data to provide confidence that this method of treatment will work while being able to use the data for building of the oral appliance.

It is proven that the advancement of the mandible allows the patients airway to open. It is also proven that the vertical position of the mandible relates to the airway. It is also proven that it is currently impossible to replicate what a sleeping patent's airway is doing while they are awake without them being asleep and in that relaxed state. This is one reason why the AMM is novel in enabling a doctor to determine the best course of treatment for apnea patients.

In some embodiments, the addition of the tongue sensor and a nasal sensor to the AMM may also be an important feature to the entire diagnosis. With Sleep Apnea anything that causes a blockage in the airway contributes to this disease. By knowing precisely the cause of the apnea allows doctors to determine either the best Oral Appliance or alternative treatment. For instance if the tongue is not in the physiological rest positions, that is, just behind the front teeth on the roof of your mouth, then this placement could be the main cause of the apnea. Once this is determined, the AMM can titrate the jaw minimally and the doctor will be able to provide the patent with an oral appliance that focuses on placing the tongue in the correct position.

The ability to monitor air intake through the nose, the location of the tongue and the ability to titrate the mandible while a person is asleep with all muscles relaxed gives the doctor the ability to determine the best course of action before anything is prescribed.

Without the proper data, it is difficult and near impossible for doctors to determine the best medical approach for the patient. This is one of the main reasons CPAP systems are preferred because a constant flow of pressurized air is constantly being breathed by the patient to keep their airway open. The alternative is to use an AMM to determine what is happening before a treatment is prescribed. It is our belief that the majority of Sleep Apnea patents will benefit from an oral appliance if that oral appliance is placed in the correct position based upon the AMM used as a diagnostic tool.

### BRIEF DESCRIPTION OF THE DRAWINGS

FIG. 1 shows a mandibular manipulator in accordance with an embodiment of the disclosure in a perspective view.

FIG. 2 shows a portion of the mandibular manipulator in a side perspective view.

FIG. 3 shows an upper palate registration for use with a mandibular manipulator in a perspective view.

FIG. 4 shows a low bite tray subassembly for use with a mandibular manipulator in a perspective view.

FIG. 5 shows a perspective view of a servomotor for use with a mandibular manipulator.

FIG. 6 shows a nasal and tongue sensors independent of mandible manipulator shown in upper perspective view.

FIG. 7 shows a nasal and tongue sensors independent of mandible manipulator shown in lower perspective view.

FIG. 8 shows a mandibular manipulator in accordance with an embodiment of the disclosure depicted within a cross section of a human head anatomy.

FIG. 9 shows positions of surface electrodes onto a neck and head human anatomy for determining tongue placement for electromyography methodology.

FIG. 10 shows positions of surface electrodes onto a human head anatomy for determining tongue placement when using capacitance methodology.

FIG. 11 shows hypopharynx volume change in habitual position and with Andra Gauge positioning mandible in optimal position

FIG. 12 shows pilot study CT scans of subject #1 before and after airway optimization

FIG. 13 Shows Hypopharynx volume change in habitual position and with Andra Gauge positioning mandible in optimal position

### DETAILED DESCRIPTION

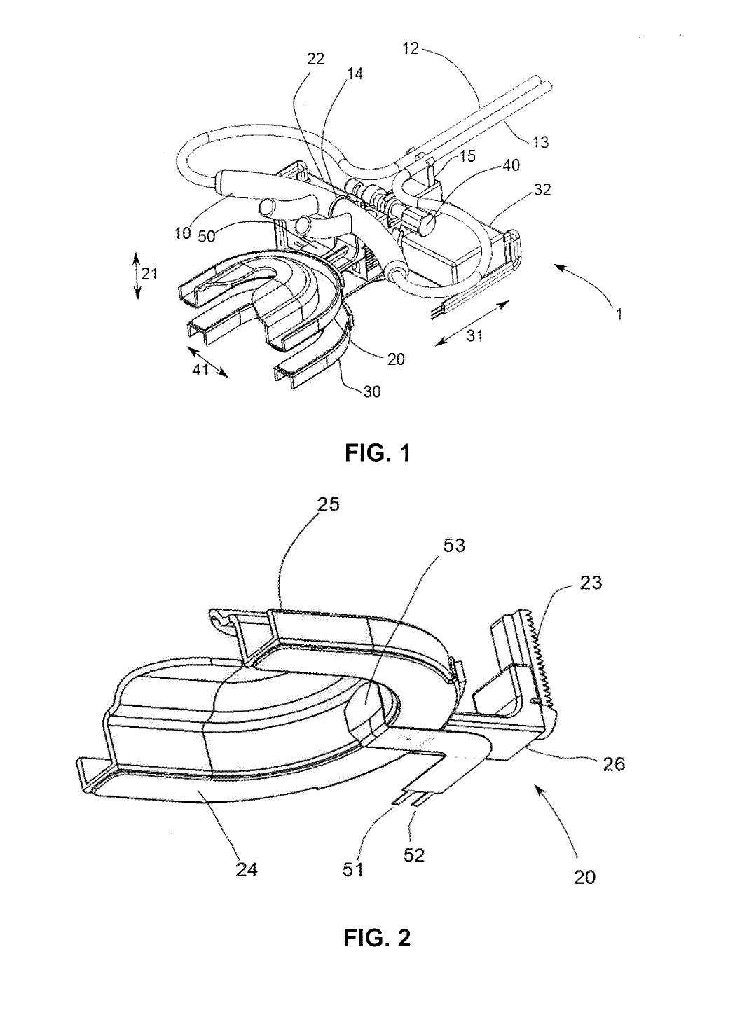

Now referring to FIG. 1, showing a top perspective view of a mandibular manipulator 1, cannula tube 10, is shown with septum 14, bifurcating the tubes both right and left. Cannula tube 10 is attached to the device by stub 15 which has cylindrical holes with slots to allow the tubes 12, 13 to snap into and temporarily remain in place.

As depicted, Upper Bite tray 20 is moved in the vertical direction 21 via motor 22 using a rack and pinion coupling. Lower Bite tray 30 is moved in the Anterior-Posterior directions 31, via motor 32 using a rack and pinion coupling. Sagittal movements 41 are created using Septum Screw 40. All of the rotational translation to linear motion of the three movements is detailed in incorporated U.S. Pat. No. 8,226,407. Although the sagittal movement shown here is created using a manual method, the present disclosure does not preclude this motion also being motor driven.

Tongue location is electrically derived using a pressure resistor 50, with the sensor located within the upper bite tray 20. The pressure sensor is similar to Pololu part #1695 available from Pololu Robotics and Electronics, 920 Pilot Road, Las Vegas, Nev. 89119.

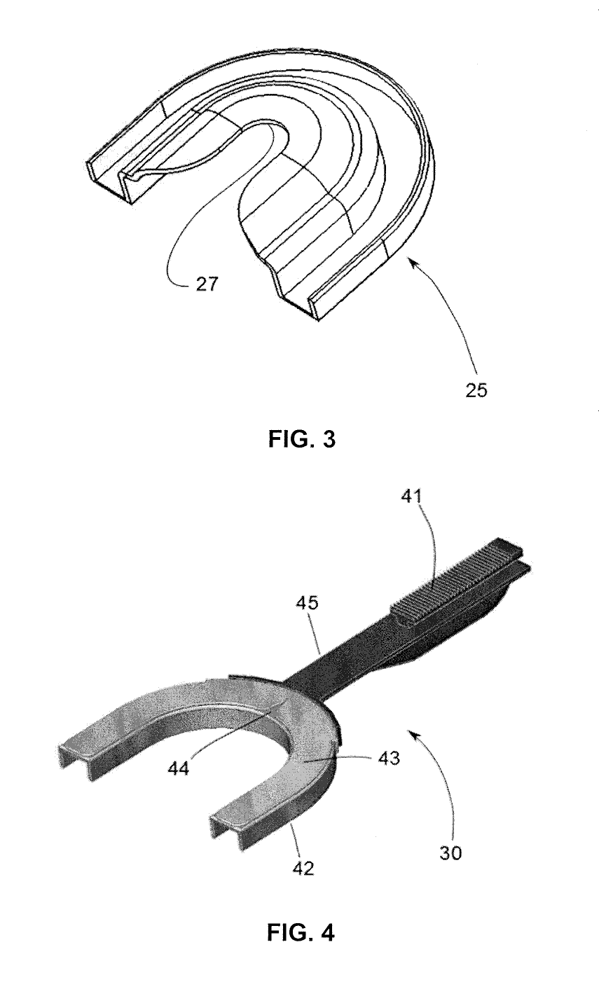

Now referring to FIGS. 2 and 3, the Upper Bite 20 is shown as disconnected from the main device. Gear rack 23, bracket 26, and bite arch 24 can be one single piece or multiple pieces assembled. In some embodiments, bite arch 24 may be metal to achieve a sufficient amount of stiffness. In FIG. 3, and upper palate retainer 25 (e.g., bite registration material), which may be formed from an acrylic material, is pre-shaped as shown, but has a modeling-clay like consistency so as to allow it to be hand shaped to the patient's upper teeth and palate. Relief slot 27 is provided to allow for the material to be widened or narrowed as necessary to best fit the patient's maxilla width. Upper palate retainer 25, is attached to bite arch 24 using adhesive. Once this hand shaping has been performed, the Upper Bite Tray 20 is removed and the upper palate retainer 25 is cured to a hard stiff material using a visible light of a fixed electromagnetic frequency. Tongue sensor 53, which may be an integral part of 50-52, is created with printed electrical traces sandwiched between two planar layers of Mylar film. This sensor 50 is attached using pressure sensitive adhesive. Electrically conductive terminals 51, 52 carry the analog resistive signal to an interconnection with the device's digital computer. Multiples of sensor 50 may also be used in a similar or same manner Although this embodiment shows a resistive pressure-sensing patch to detect if the patient's tongue is in a position expected during sleep, a pneumatic tube routed to this approximately location, left with an open aperture that the tongue covers in its normal position and connected to an electrical transducer, will produce the same result.

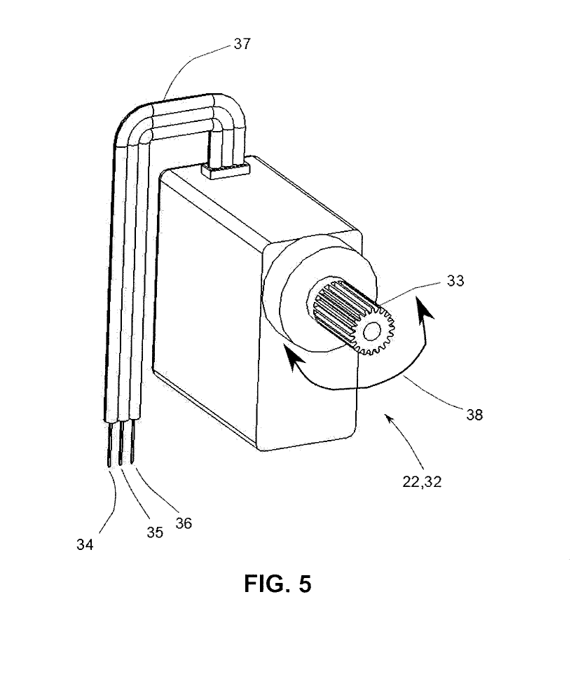

Now considering FIGS. 4 and 5, the lower bite tray 30 and common servomotor 22, 32 are shown, respectively. The lower bite tray 30 comprises gear rack 41, bite arch 43, and connecting bridge 45. These pieces may be made and assembled separately or completed as a single unit. In some embodiments, bite arch 43, may be made of a metal material to allow for sufficient stiffness. It should be noted that slot 44, is intended to allow for a small planar hinging of the bite arch to accommodate patients with a wider mandible. The upper bite plate 24 (FIG. 2) has a similar (e.g., identical) slot. As depicted in FIG. 5, gear rack 41 engages pinion 33, of motor 22, 23 to change rotational motion to linear motion. The motor 22, 32 is attached in this embodiment through threaded fasteners or adhesives. The motor is common to the two axes of the embodiment and is powered by cable 37. Cable 37 including conductors 34, 35, 36 provide supply and return power along with motor position data to the computer. In some instances, there may be more conductors within the cable depending on the type of motor utilized. Motor 22, 23 can rotate gear 33 in direction 38.

In some embodiments, and similar to the upper palate retainer 25, the lower bite retainer 42 (e.g., bite registration material), which is adhered to bite arch 43, may be formed from an acrylic material that is intended to be hand shaped to the patient's lower teeth, removed from the patient's mouth, and cured in a specific electromagnetic wavelength of light. This curing causes the hand-shaped lower bite retainer 42 to become a temporary oral appliance allowing for intimate contact with the teeth such that, in manipulating the mandible, there is little or no play between the teeth and the bite trays.

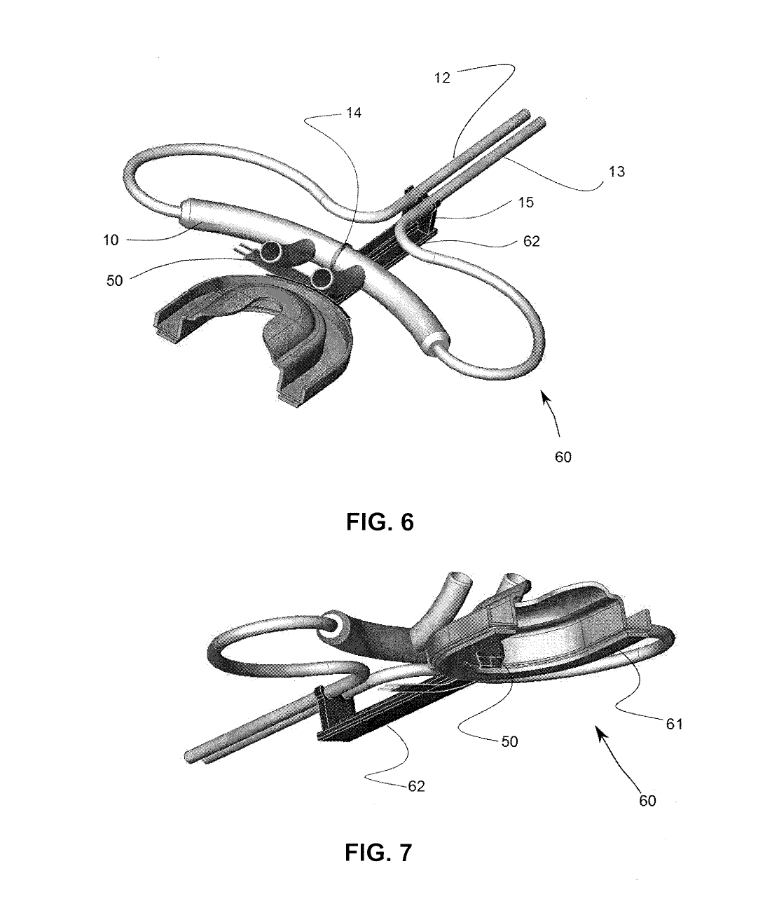

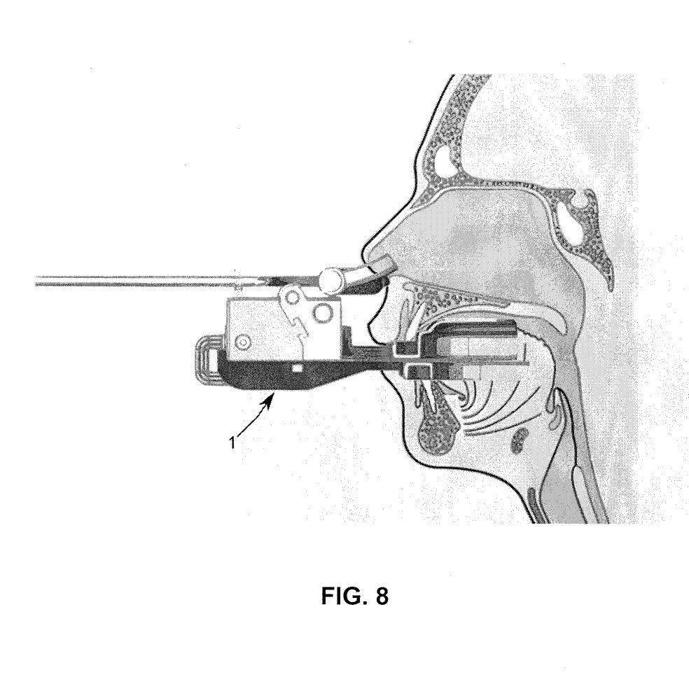

Referring now to FIGS. 6 and 7, there are shown perspective views top and bottom of a device 60, with only its bilateral nasal and tongue placement sensors. In this embodiment, the manipulation of the mandible is not the focus, but the nasal flow and tongue placement are. Considering FIG. 6, the device 60 comprises a frame consisting of a bite fork 43, bridge 60, bilateral nasal cannula 50, and tongue placement sensor 50. Stub 15, as described earlier in FIG. 1, provides a snap-in feature for holding air tubes 12, 13. In FIG. 7, rubber arch 60 is attached to the bite fork to prevent damage or discomfort to the patient's lower teeth. The construction of device 60 is similar to descriptions of the current disclosure except for the removal of the mandible manipulation features. In another embodiment, bilateral nasal cannula 10, and tongue placement sensor 50 can be attached to the patient using a cannula lanyard and sensor 50 can be temporarily adhered to the preferred location within the patient's mouth, all without the frame of device 60. Referring to FIG. 8, a mandibular manipulator 1 is shown in cross section in its application to a patient's oral cavity.

In some embodiments, the device 60 may include an additional airflow sensor positioned in front of the subject's mouth.

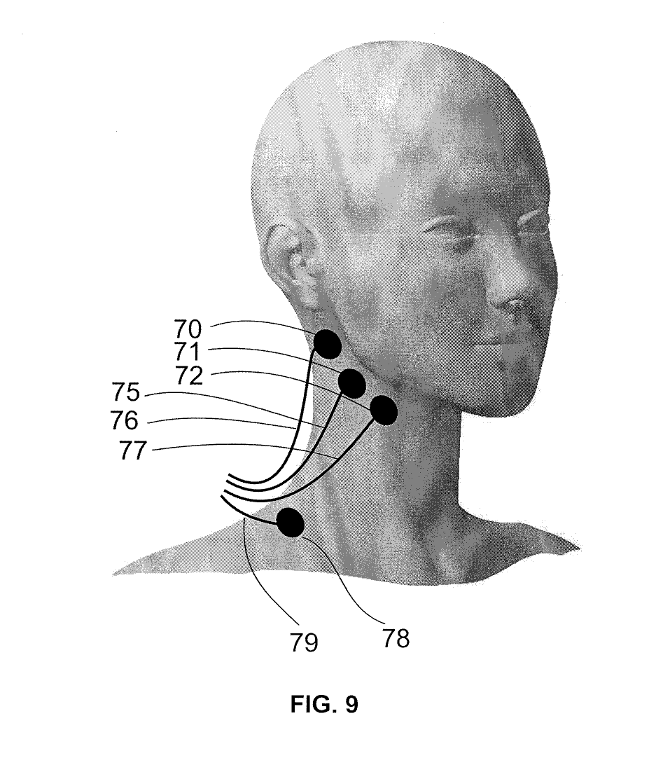

Referring now to FIG. 9, shows the approximate locations of surface electrodes 70, 71, 72 along with their corresponding wires 75, 76, 77 which lead to a buffer amplifier and then electromyography recording instrument for another instantiation of tongue position location. Surface electrode 78 and corresponding wire 79 are shown in approximate position to provide a ground potential for the electromyography circuit. The electrical potential of the tongue's extrinsic muscles (e.g., genioglossus muscle, protrudes the tongue as well as depressing its center; hyoglossus muscle, depresses the tongue; styloglossus muscle, elevates and retracts the tongue) may be measured with the sensors attached to the upper neck position on the patient. While all of these muscles could be monitored simultaneously, only one set is shown monitored in FIG. 9.

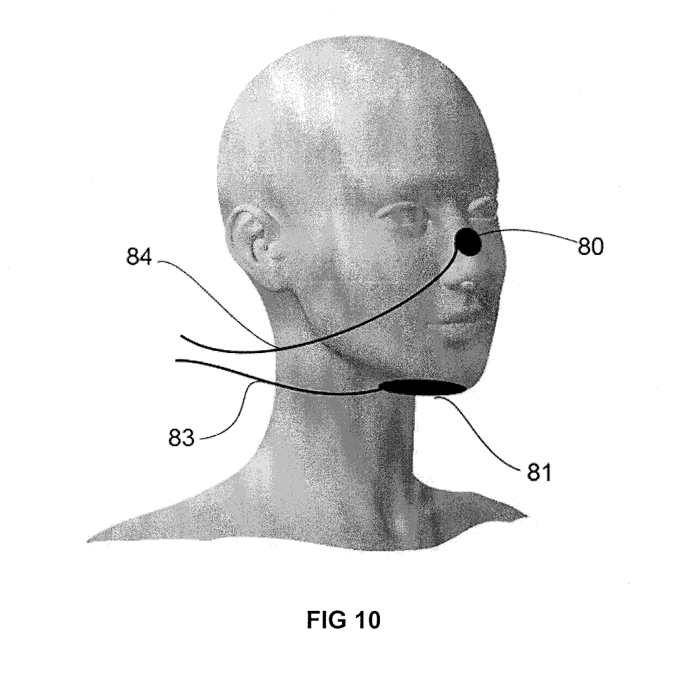

Referring now to FIG. 10, shows the approximate location of surface electrodes 80, 81 along with their corresponding wires 83, 84 that connect to an electrical capacitance measuring circuit in another instantiation of a tongue position location sensing method.

Once being apprised of the instant device and methods, one of ordinary skill in the art will be able to make and use the device.

### EXAMPLE

The assignee of the present application, Kosmo Technologies LLC, engaged in a pilot study of 10 sleep apnea patients to test a new technique in the fitting of oral appliances. Currently, fitting an oral appliance to treat sleep apnea is at best a trial and error method leaving the Sleep Physician with a lack of confidence in using this treatment approach.

The study was a pretest-posttest design that had the following goals:

1. Research a reproducible method of capturing the optimal mandibular position for oral appliance therapy by working with both the sleep physician as well as the dentist.

2. Demonstrate that vertical as well as protrusion of the mandible is important for significantly opening the hypopharynx airway using a millimeter incremental approach referred to as the Step Back Technique. This is proven by measurements produced through a series of before and after CT scans.

3. Using the Step Back Technique as a means of creating oral appliances and resulting efficacy, we demonstrated the collaborative chronic care model for obstructive sleep apnea across the sleep specialty in collaboration with dental providers.

4. The study will perform comparisons of AHI (Apnea Hypoxia Index) to demonstrate before and after treatment results.

Mild-to-moderate obstructive sleep apnea (OSA) has been shown to be treatable successfully by oral appliance therapy (OAT) versus continuous positive airway pressure (CPAP). Through a number of studies and their reviews over the past 20 years, OST has been shown to be successfully implemented using both protrusion and vertical measurements. However, in nearly all of these studies, the predictors of both protrusion and vertical have not always been consistent. Because of that distinction, and because no model exists for their direct role, sleep physicians have not had the complete confidence in OAT for their patients. Dentists also know that in many cases protrusion will pull the tongue forward to prevent OSA but that too much protrusion can cause TMJ disorder, changes in bite, and other side effects. Anecdotally, dentists will tell you that some patients respond very well to OAT with simple protrusion (that includes inferred vertical position due to the nature of the temporomandibular joint geometry) but in other instances where a patient does not respond to OAT the dentist cannot explain why improved results are not achievable.

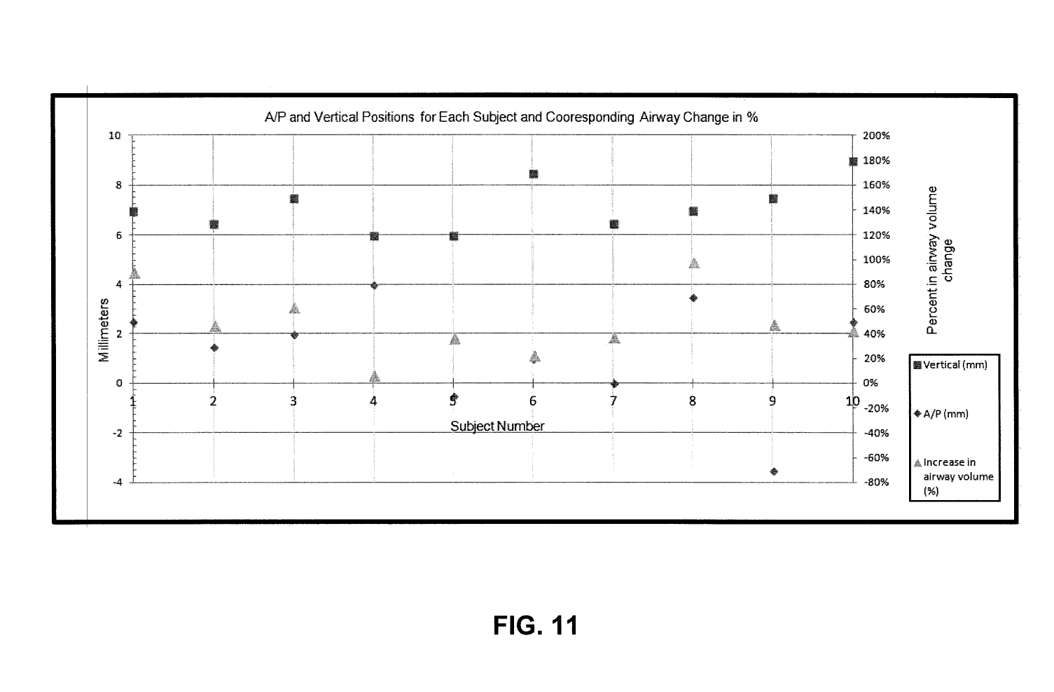

In this multi-disciplinary study, a team led by a board certified sleep physician and partnered with a dentist, performed a regimen referred to as the Step Back Technique using the Andra Gauge (U.S. Pat. No. 8,226,407) and a snore sound test. This research is a privately funded proof of concept pilot study in which 10 apnea patients were shown to have improved airway openings using the step back technique and confirmed with a Kodak 9500 cone beam computer tomography (CT) scanner. The pilot study was organized and self-funded by Kosmo Technologies, LLC, Salt Lake City, Utah. FIG. 11 shows subjects' airway changes at their hypopharynx, first in their habitual position and then with the Andra Gauge positioning their mandible from the outcome of the snore sound test.

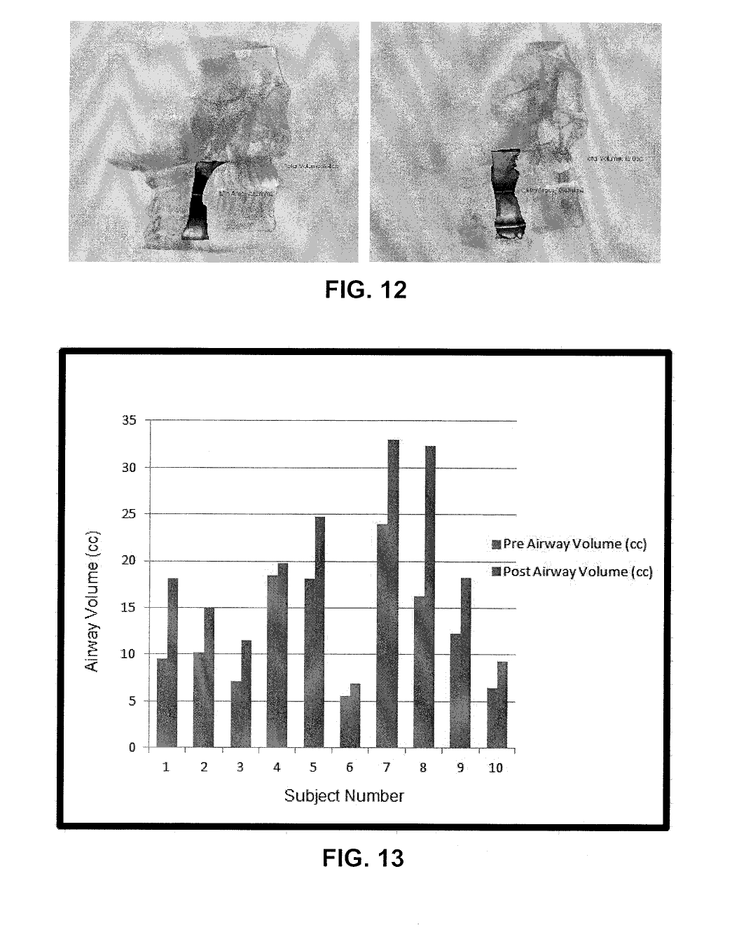

FIG. 12 is a set of CT scan depictions of subject #1 from the habitual seated position versus seated position with the Andra Gauge in place with the patience's optimal mandible position.

FIG. 13 shows the amount of airway volumetric change for each subject pre and posttest.

A second part of this pilot study is in progress in which the 10 original subjects are being provided oral appliances with the Andra Gauge settings from pilot study #1. Once these OA's are fitted, a second comparative polysomnography testing will be performed to compare with the subjects' original polysomnography data.

This study was conducted in two parts. The first part tested each subject using a new approach named the step back technique to optimize the subject's airway by manipulating the mandible position. The second part of the study prescribes each subject with an oral appliance. With each subject using their oral appliance, follow up polysomnography testing will be performed. The result of the second polysomnography tests is then compared with each subject's initial polysomnography test to determine if AHI scores have changed.

The step back technique protocol is described as follows:

The Andra Gauge™ (AG) is used in this study because it is the only device that will allow a doctor to manipulate the mandible in all three Axis (A/P, Vertical and Sagittal) while also maintaining the patient's mandible in that position for testing purposes. The hypothesis of this study was to indicate, either in a positive or negative way, that the protocol described below provides an effective tool for dentists and sleep physicians to use in setting the mandible in the optimal position for treating sleep apnea with Oral Appliance Therapy (OAT).

At all steps in this procedure, the patient was asked if they are comfortable or experiencing any discomfort. The traditional medical question of patient pain will be asked on a scale of 0-10 with 10 being the least comfortable. This scale will be referred to as “the comfort scale.” The patient was asked to use their fingers as a communication method so that the AG does not have to be removed from the patient's mouth during the procedure. This was based upon the reasoning that if the OA is not positioned in a location that the patient will view as comfortable then patient compliance will not be as high. The Andra Gauge will allow for various mandibular positions allowing for a position that will be both comfortable as well as optimal for increased airway.

The steps of the procedure included:

1. Place the subject in a supine position or in the most reclined position similar to the sleeping position of the patient. The supine position relates closely to a sleep like position while resulting in some prolapse of the airway causing a further decrease in its size. This can impede airflow during respiration and can cause increase in snoring.

2. Once the patient is in the inclined position, ask the patient to relax as much as possible. The patient will then be asked to try to make a snoring sound while breathing through their mouth. Proceed to place the Andra Gauge into the patient's mouth. In optimizing the airway, set the centric position of the Andra Gauge at zero. Once the centric is set, have the patient move their mandible forward or anteriorly using the AG as a support and measuring tool until the patient can no longer make the snoring sound or there is an audible or otherwise noticeable opening in the airway. This is often times an uncomfortable position and places stress on the TMJ condyles and surrounding tissues. Ask the patient during this procedure to express if they are experiencing any pain or discomfort. On a scale of 0 to 10, inquire with the patient how much pain or discomfort they are experiencing. If this pain or discomfort reaches a level of 5, and they are still making the snoring sound, stop and proceed to the next step.

3. Once the patent has stopped snoring or has reached a discomfort level of 5, move the mandible backwards or posterior 1 mm while also increasing the vertical, 1 mm. At each interval, ask the patient to make a snoring sound. This pattern of 1 mm posterior movement and 1 mm vertical movement continues with the patient being asked each time to make a snoring sound. Depending upon your judgment of the patient's comfort level you can also lock the A/P into position and slowly increase the patient's vertical in 1 mm increments. Often times 0.5 mm will make a significant difference as the best position is converged upon.

4. Once a position is found where a patient stops being able to make the snoring sound, or the snoring sound is minimized, or the airway has audibly opened, ask the patient if they are comfortable using the comfort scale. If the patient is comfortable and can no longer make a snoring sound or that the snoring sound is reduced, or the airway has audibly opened, lock the positions of the AG, remove the AG from the patient's mouth and record the positions of the AG on the data sheet.

5. If the patient is not comfortable then try increasing the vertical and moving the mandible more anterior using 1 mm increments in both directions each time. There should be a point in which the patient is comfortable and can no longer make the snoring sound or that the snoring sound is reduced, or the airway has audibly opened. Once this has been accomplished, the AG should be locked using its locking screws and the positions recorded on the data sheet.

6. Patient is now ready for CT scan.

The CT scan provided a means of quantifying the measurement near the hypopharynx. In the pilot study, this area was found to be the narrowest and showed the most improvement in volume with mandible repositioning. CT scans will be made with the subject in the habitual position and then with their mandible held in position by the AG.

The second part of the study was to take upper and lower bite arch impressions of each subject by the dentist. The dentist also created a dental bite using the AG with the vertical and A/P positions recorded from the first part of the study. An oral appliance was prescribed using these bite registrations for each test subject. The oral appliance received a final fit to the patient by the dentist and then released back to the sleep physician. After at least a week of using the oral appliance, the patients were scheduled for a follow up polysomnography testing.

Results expected from this research are hinted at by the pilot study and studies akin to this where both the A/P and vertical mandible position are made relative to one another. We expected an outcome where the A/P protrusion is reduced and the vertical is larger than what would normally be expected. FIG. 13 is data from the pilot study indicating each subjects A/P and vertical mandible positions along with the percent increase in respective airway volume measured by the CT scan images.

It was also expected that AHI levels will be reduced as a result of the data found in the citation documents and based upon comparison with our pilot data.

The significance of this research was to develop a protocol by which a sleep physician working with a dentist can optimize the airway using an oral appliance to treat mild to moderate sleep apnea. There are currently several “schools” as to determining mandible position for oral appliance therapy, but none that provide the confidence that a sleep physician needs to help prescribe OAT more often or in cases where the patient is non-compliant with CPAP or prefers another therapy over CPAP. In some cases, CPAP and OAT can be used together to reduce the CPAP pressure to a tolerable level.

What is claimed is:

1. A motorized mandibular manipulator for adjusting a mandible of a subject in at least one axis to diagnose health problems pertaining to the mandible, the mandibular manipulator comprising:
an upper bite fork attached to a first sliding gear rack and a lower bite fork attached to a second sliding gear rack;
at least one motor for driving the first sliding gear rack and the second sliding gear rack, wherein:
the at least one motor being configured for rotational movement;
the at least one motor being controlled by a computer;
the at least one motor comprising a feedback feature for communicating at least one position of the at least one motor to the computer; and
the at least one motor being controlled by computer software adapted for controlling the at least one motor manually through the computer or automatically through a software algorithm.

2. The motorized mandibular manipulator of claim 1, at least one tongue sensor for communicating a subject's tongue position to the computer.

3. The motorized mandibular manipulator of claim 2, wherein the at least one tongue sensor comprises at least one of a resistive pressure pad and at least one vacuum or pressure tube with an orifice communicating with a transducer.

4. The motorized mandibular manipulator of claim 2, wherein the at least one tongue sensor comprises a sensor configured to be adhered to the subject's hard palate.

5. The motorized mandibular manipulator of claim 2, wherein the at least one tongue sensor comprises a sensor configured to measure capacitance of human tissue between an external surface of the subject's nose and a lower part of the subject's chin to determine tongue position.

6. The motorized mandibular manipulator of claim 2, wherein the at least one tongue sensor comprises a sensor configured to measure electrical pulses from nerves controlling the subject's tongue to determine tongue position.

7. The motorized mandibular manipulator of claim 1, further comprising a bilateral nasal cannula tube positioned and configured to be placed at least partially within at least one nostril of the subject, the bilateral nasal cannula tube communicating with a transducer and the computer for determining nasal flow from the at least one nostril.

8. The motorized mandibular manipulator of claim 7, further comprising an additional airflow sensor positioned in front of the subject's mouth.

9. A mandibular manipulator for adjusting a mandible of a subject in at least one axis to diagnose health problems pertaining to the mandible, the mandibular manipulator comprising:
an upper bite fork attached to a first sliding gear rack and a lower bite fork attached to a second sliding gear rack; and
at least one tongue sensor for communicating a subject's tongue position to the computer.

10. A mandibular manipulator for adjusting a mandible of a subject in at least one axis to diagnose health problems pertaining to the mandible, the mandibular manipulator comprising:
an upper bite fork attached to a first sliding gear rack and a lower bite fork attached to a second sliding gear rack; and
a bilateral nasal cannula tube positioned and configured to be placed at least partially within at least one nostril of the subject, the bilateral nasal cannula tube communicating with a transducer and the computer for determining nasal flow from the at least one nostril.

11. A dual sensor diagnostic device for diagnosing health problems that can determine bilateral nasal flow and tongue position, the dual sensor diagnostic device comprising a rigid bite fork covered with a malleable plastic resin material configured to be cured to a rigid state by a specific frequency of light, at least one tongue sensor for communicating a subject's tongue position to a computer, a bilateral nasal cannula tube positioned and configured to be placed at least partially within each nostril of the subject, the bilateral nasal cannula tube communicating with a transducer and the computer for determining nasal flow from each nostril.

12. The dual sensor diagnostic device of claim 11, wherein the at least one tongue sensor comprises at least one of a resistive pressure pad and at least one vacuum or pressure tube with an orifice communicating with a transducer.

13. The dual sensor diagnostic device of claim 12, wherein the at least one tongue sensor comprises a sensor configured to be adhered to the subject's hard palate.

14. The dual sensor diagnostic device of claim 12, wherein the at least one tongue sensor comprises a sensor configured to measure capacitance of human tissue between an external surface of the subject's nose and a lower part of the subject's chin to determine tongue position.

15. The dual sensor diagnostic device of claim 12, wherein the at least one tongue sensor comprises a sensor configured to measure electrical pulses from nerves controlling the subject's tongue to determine tongue position.

16. The dual sensor diagnostic device of claim 12, further comprising an additional airflow sensor positioned in front of the subject's mouth.

17. A system comprising:
the motorized mandibular manipulator of claim 1; and
a computer readable medium having instructions stored thereon that, when executed by a processor, causes the processor to:
operate the at least one motor of the motorized mandibular manipulator; and
receive and analyze input data representing a physiological state of the subject during a sleep study to find an optimum mandible position to reduce an incidence of sleep apnea.

18. A system comprising:
the motorized mandibular manipulator of claim 1; and
a computer readable medium having instructions stored thereon that, when executed by a processor, causes the processor to:
operate the at least one motor of the motorized mandibular manipulator; and
receive and analyze feedback data from the at least one motor to find an optimum mandible position to reduce an incidence of sleep apnea.

19. A system comprising:
the motorized mandibular manipulator of claim 1; and
a computer readable medium having instructions stored thereon that, when executed by a processor, causes the processor to:
operate the at least one motor of the motorized mandibular manipulator;
receive and analyze feedback data from the at least one motor to determine and quantify mandible muscle resistance from the subject's mandible; and
report the quantified mandible muscle resistance.

20. A method of forming an oral appliance, the method comprising:
determining a mandible position to reduce an incidence of sleep apnea for a subject by adjusting the motorized mandibular manipulator of claim 1 in three independent axes of movement; and
utilizing the mandible position determined by the motorized mandibular manipulator to construct of an oral appliance for the specific sleep apnea patient tested.
